(19) United States
(12) Patent Application Publication
(10) Pub. No.: US 2012/0108993 A1
(43) Pub. Date: May 3, 2012

(54) ELECTRODE SHAPES AND POSITIONS FOR REDUCING LOSS OF CONTACT IN AN IMPLANTABLE ECG RECORDER

(75) Inventors: Charles R. Gordon, Phoenix, AZ (US); Brian B. Lee, Golden Valley, MN (US); Michael W. Barror, Gilbert, AZ (US)

(73) Assignee: Medtronic, Inc., Minneapolis, MN (US)

(21) Appl. No.: 13/282,167

(22) Filed: Oct. 26, 2011

Related U.S. Application Data
(60) Provisional application No. 61/407,204, filed on Oct. 27, 2010.

(51) Int. Cl. A61B 5/042

(52) U.S. Cl. 600/509

(57) ABSTRACT

An improved design for subcutaneous monitors that addresses the problem caused by bubbles of air may remain in the pocket in which the device is implanted. As implantable monitors and their associated electrodes are reduced in size, these bubbles may in some cases cover one or both electrodes, interfering with sensing of the ECG signal. The invention addresses this problem by configuring the electrodes to increase the pressure of the electrodes against the tissue above the electrodes relative to the pressures exerted by the adjacent outer facing surfaces of the device.

### CROSS-REFERENCE TO RELATED APPLICATIONS

This application claims the benefit of U.S. Provisional Application No. 61/407,204, filed on Oct. 27, 2010. The disclosure of the above application is incorporated herein by reference in its entirety.

### BACKGROUND

This invention relates to an implantable monitoring device for sensing physiologic events with minimally invasive intrusion into an animal or patient body, and is particularly well suited for long term monitoring of body events like ElectroCardioGrams (ECG's) and in monitoring other body physiologic events. By enabling easy monitoring and recording of physiologic events in the patient's body, such events can then be studied at leisure outside the body, providing research, diagnostic and therapeutic opportunities not otherwise available.

Some currently available implantable subcutaneous ECG recording systems employ ECG electrodes located on an outward-facing, generally flat surface area of the device. The electrodes are so located in order to reduce motion artifacts from the surface of the muscles below. In some cases, these devices may suffer from loss of signal, which can be a source of falsely detected asystoles, particularly in the first week or two after implant.

Examples of prior subcutaneous ECG recording systems are disclosed in U.S. Pat. No. 5,331,966 for a “Subcutaneous multi-electrode sensing system, method and pacer”, by Bennett, et al, U.S. Pat. No. 5,987,352 for a “Minimally invasive implantable device for monitoring physiologic events”, filed by Klein, et al. and U.S. Pat. No. 7,035,684 for a “Method and apparatus for monitoring heart function in a subcutaneously implanted device”, by Lee, all of which are incorporated herein by reference in their entireties.

### SUMMARY OF THE INVENTION

The present invention is directed toward reducing the loss of signal described above. The inventor has determined that bubbles of air can remain in the subcutaneous pocket in which the device is implanted. These bubbles may in some cases cover one or both electrodes, interfering with sensing of the ECG signal. As implantable monitors and their associated electrodes are further reduced in size, the potential for this problem to occur correspondingly increases. For this reason, the present invention is believed particularly desirable for device having very small volumes, for example less than three cubic centimeters, and particularly for devices less than one and a half cubic centimeters.

The invention addresses this problem by configuring the electrodes to increase the pressure of the electrodes against the tissue above the electrodes relative to the pressures exerted by the adjacent outer facing surfaces of the device. This result may be accomplished using one of several approaches. A first embodiment provides raised surfaces for the electrodes, so that the contact pressure is enhanced where the inner surface of the skin contacts the electrodes, typically in areas at or adjacent to the area where the skin curves or folds over the outward facing edge and/or end surfaces of the device. This approach is particularly amenable to electrodes deposited in layers on an insulative substrate material such as ceramic.

A second embodiment accomplishes a similar result by placing the electrodes on the ends of the outward facing halves of the device, covering part of the edge and/or end surfaces of the device while maintaining a distance from the back-side of the device in order to reduce susceptibility from motion artifacts due to the movement of the underlying tissue, such as the muscle fascia in some implants. Generally these devices will have curved end and/or side surfaces comfort, so that the electrodes are correspondingly curved, maximizing surface area.

A third embodiment employs electrodes which covers end portions of the device including both the inward and outward facing surfaces, which has the added benefit of maintaining signal despite the device flipping over, for example due to the patient suffering from “Twiddlers Syndrome”. This third embodiment maintains a large area of surface contact and pressure against the inward and outward facing encompassing tissue. In this embodiment, the potential problem of muscle noise or motion artifacts motion artifacts due to proximity of the electrodes to the underlying muscle tissue from contact below is may be overcome by some other method. For example, the device may be implanted above a layer of fat just under the dermis. In such locations, the performance can be as good as or better than when using electrodes located only on the outward facing portions of the device.

### BRIEF DESCRIPTION OF THE DRAWINGS

The following drawings are illustrative of particular embodiments of the present invention and therefore do not limit the scope of the invention. The drawings are intended for use in conjunction with the explanations in the following detailed description. Embodiments of the present invention will hereinafter be described in conjunction with the appended drawings, wherein like numerals denote like elements.

FIG. 1 illustrates top, end and side views of a first embodiment of the invention.

FIG. 2 illustrates top, end and side views of a second embodiment of the invention.

FIG. 3 illustrates top, end and side views of a third embodiment of the invention.

FIG. 4 illustrates two alternative end views of the device of FIG. 1.

### DETAILED DESCRIPTION

The following detailed description is exemplary in nature and is not intended to limit the scope, applicability, or configuration of the invention in any way. Rather, the following description provides practical illustrations for implementing exemplary embodiments of the present invention.

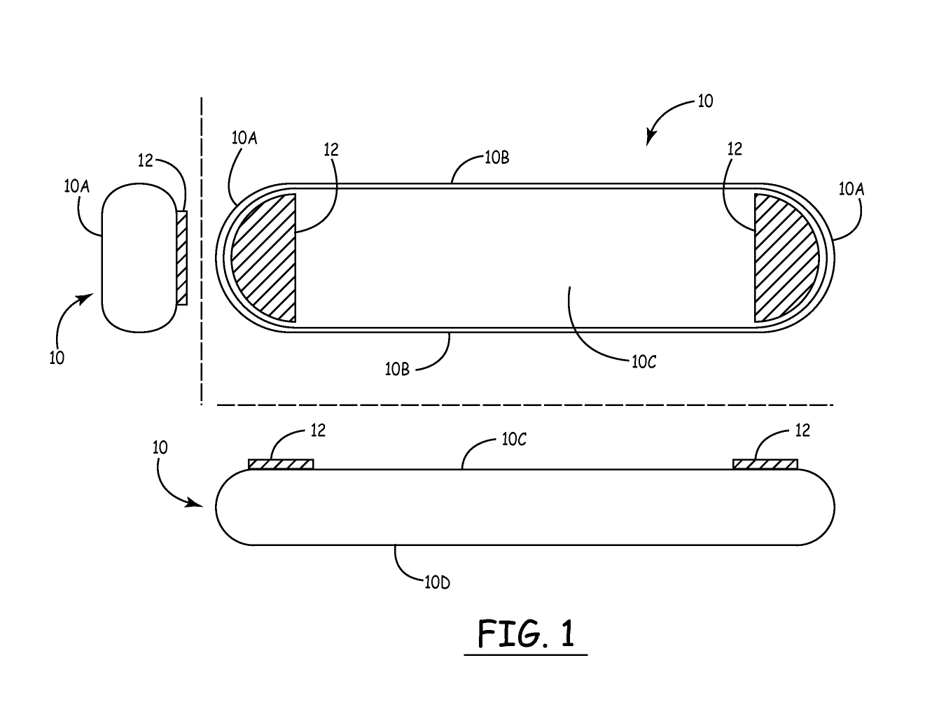

FIG. 1 is a representation of an implantable medical device (IMD) 10 that may be used in accordance with certain embodiments of the invention. The device may be any device that is capable of measuring hemodynamic parameters (e.g., blood pressure signals) from within a ventricle of a patient's heart, and which may further be capable of measuring other signals, such as the patient's electrogram (EGM).

The internal circuitry and other functional components of the device may correspond generally to those described in the above-cited Klein, et al, Bennett, et al. and/or Lee patents, incorporated herein by reference. The circuitry typically includes circuitry for monitoring ECG signals, storing them in memory ant transmitting them to an external monitor. In most embodiments, it is anticipated that the device will also include circuitry for receiving commands from external devices and modifying its operation in accordance with those control signals.

The volume of the device may be three cubic centimeters or less, preferably 1.5 cubic centimeters or less, and the general configuration may be as illustrated. The device's configuration as illustrated is an elongated, flattened configuration with rounded edge surfaces (10B) and end surfaces (10C).

The rounded edge (10B) and end (10C) surfaces reduce discomfort and irritation to the patient's tissues. The rounded end surfaces in particular facilitate subcutaneous introduction of the device by means of an introducer set, for example as described in US Patent Application Publication No. 20090036917A1 for “tools and method for implanting a subcutaneous device”, filed by Anderson or US Patent Application Publication No. 20100094752A1 for a “Subcutaneous Delivery tool”, Filed by Wengreen, et al., both of which are hereby incorporated by reference in their entireties. The flattened configuration assists in preventing the device from flipping over after implant.

As illustrated, the device has generally flat outward facing (10C) and inward facing (10D) surfaces. In the first embodiment as illustrated, the outward facing surface 10C takes the form of a ceramic or other insulative substrate upon which conductive electrodes 12 are deposited. Electrodes 12 may be deposited in multiple layers using photolithographic or other techniques of the sort widely used to deposit conductive material onto ceramic or other conductive substrates, for example as described in U.S. Pat. No. 6,564,106 for “Thin film electrodes for sensing cardiac depolarization signals”, filed by Guck, et al. or U.S. Pat. No. 6,631,290 for “Multilayer ceramic electrodes for sensing cardiac depolarization signals:, also filed by Guck, et al., both hereby incorporated herein by reference in their entireties.

The outward facing surface 10C may be formed of a ceramic or other non-conductive substrate applied to or included as part of the device enclosure, as disclosed in US Patent Application Publication No. 2003012320A1, for an “Implantable medical device having a housing or component case with an insulative material formed thereon, and methods of making same”, filed by Solom or U.S. Pat. No. 5,470,345 for a “Device with multi-layer ceramic enclosure”, by Hassler, et al, both of which are also hereby incorporated herein by reference in their entireties. Connections between the electrodes 12 and the circuitry within the devices may be made according to any of the previously listed references.

In the side view and end view of FIG. 1, it can be seen that the exposed outward facing surfaces of electrodes 12 extends outward slightly from the outward facing surface 10C. As discussed above, the outward extension and location of the electrodes of this embodiment of the device will tend to exert higher pressure against the inner surface of the skin or other overlying tissue than does the adjacent relatively flattened outward facing surface of the device. As a result, bubbles which might form in the pocket are less likely to accumulate between the outward surfaces of the electrodes and the overlying skin or other tissue.

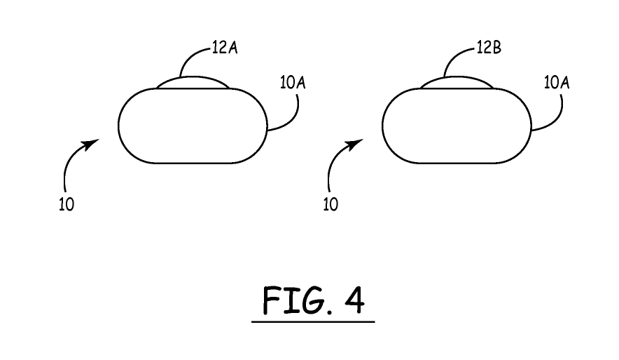

While the outer surface of the electrodes 12 as illustrated are generally flat, they may instead be made to have an outwardly curved, peaked or domed configurations by either depositing the conductive material in layers of different outer circumferences or by depositing the layers on pre-formed curved portions of surface 10C. Exemplary alternative configurations are illustrated in FIG. 4, showing end views of the device 10 carrying domed (12A) and curved (12B) versions of the electrodes as so manufactured.

The electrodes themselves may be made of any biocompatible conductive materials, for example including those listed in the above-cited Bennett, Klein, Lee and Guck patents and applications. Portions of the device housing other than the non-conductive substrate may be manufactured of any biocompatible material, including biocompatible metals such as stainless steel and titanium as well as of biocompatible plastics such as epoxies, silicone rubber, polyurethanes, and the like. In some embodiments, the device may include both metal and plastic components, generally as disclosed in the above-cited Klein, et al patent.

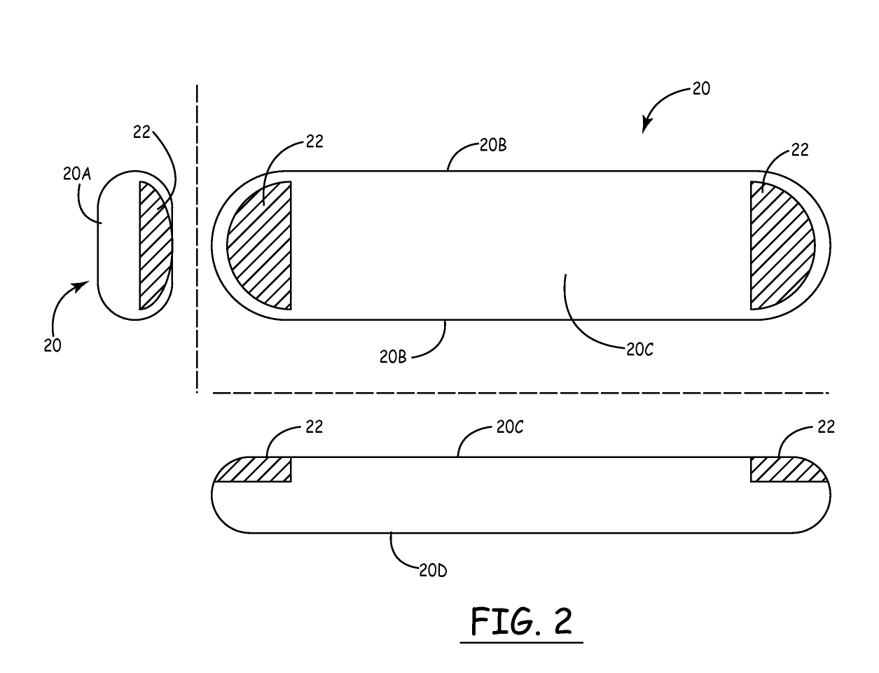

FIG. 2 is a representation of an implantable medical device (IMD) 20 that may be used in accordance with certain alternative embodiments of the invention. The device similarly may be any device that is capable of measuring hemodynamic parameters (e.g., blood pressure signals) from within a ventricle of a patient's heart, and which may further be capable of measuring other signals, such as the patient's electrogram (EGM).

The internal circuitry of the device and other functional components of the device may also correspond generally to those described in the above-cited Klein, et al, Bennett, et al. and/or Lee patents, incorporated herein by reference. The volume of the device may similarly be three cubic centimeters or less, preferably 1.5 cubic centimeters or less, and the general configuration may be as illustrated. The device's over-all configuration corresponds to that of the device illustrated in FIG. 1, i.e. an elongated, flattened configuration with rounded edge (20B) and end (20A) surfaces along with generally flat outward facing (20C) and inward facing (20D) surfaces.

Portions of the device housing may be manufactured of any biocompatible material, including biocompatible metals such as stainless steel and titanium as well as ceramics and biocompatible plastics such as epoxies, silicone rubber, polyurethanes, and the like. The electrodes 22 may be fabricated of any conductive biocompatible material, as described in any of the above cited Bennett, Lee, Klein, Guck patents.

An electrode 22 may be located on a conductive portion of the housing of device 20. If so, as in the above cited Bennett and Klein patents, it will be insulated from the housing by means of a biocompatible insulative material as described therein. If located on a non-conductive portion of the housing, it may be simply attached to the non-conductive material, also as disclosed in the above-cited Bennett and Klein patents. Connection of the electrodes to the circuitry within the housing of the device 20 may be as discussed above in conjunction with FIG. 1.

In the illustrated embodiment, the electrodes are not limited to the flattened, outward facing surface but extend onto the outward facing portions of the rounded edge (20B) and/or end (20A) surfaces so that the electrode has a three dimensional curved configuration. As discussed above, the curvature and location of the electrodes of this embodiment of the device will tend to exert higher pressure against the inner surface of the skin or other overlying tissue than does the adjacent relatively flattened outward facing surface of the device. Because of this, bubbles which might form in the pocket are less likely to accumulate between the outward surfaces of the electrodes and the overlying skin or other tissue.

While not illustrated in some embodiments the portions of the electrodes 22 overlying the generally flat outward facing surface 20C of the device may also be rounded or domed as generally illustrated in FIG. 4.

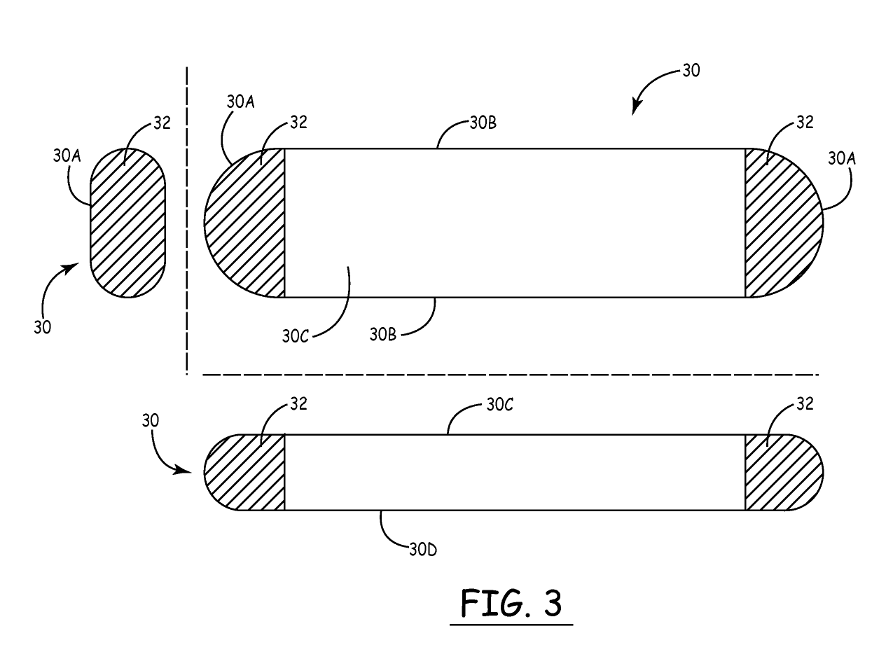

FIG. 3 is a representation of an implantable medical device (IMD) 30 that may be used in accordance with certain alternative embodiments of the invention. The device similarly may be any device that is capable of measuring hemodynamic parameters (e.g., blood pressure signals) from within a ventricle of a patient's heart, and which may further be capable of measuring other signals, such as the patient's electrogram (EGM). The over-all configuration of the device generally corresponds to the devices of FIGS. 1 and 2.

The internal circuitry of the device and other functional components of the device may also correspond generally to those described in the above-cited Klein, et al, Bennett, et al. and/or Lee patents, incorporated herein by reference. The volume of the device may similarly be three cubic centimeters or less, preferably 1.5 cubic centimeters or less, and the general configuration may be as illustrated. The device's over-all configuration corresponds to that of the device illustrated in FIG. 1, i.e. an elongated, flattened configuration with rounded edge (30B) and end (30A) surfaces; along with generally flat outward facing (30C) and inward facing (30D) surfaces.

Portions of the device housing may be manufactured of any biocompatible material, including biocompatible metals such as stainless steel and titanium as well as ceramics and biocompatible plastics such as epoxies, silicone rubber, polyurethanes, and the like. The electrodes 32 may be fabricated of any conductive biocompatible material, as described in any of the above cited Bennett, Lee, Klein, Guck patents.

An electrode 32 may be located on a conductive portion of the housing of device 20. If so, as in the above cited Bennett and Klein patents, it will be insulated from the housing by means of a biocompatible insulative material as described therein. If located on a non-conductive portion of the housing, it may be simply attached to the non-conductive material. Connection of the electrodes to the circuitry within the housing of the device 30 may be as discussed above.

In the illustrated embodiment, the electrodes are not limited to the flattened, outward facing surface but extend circumferentially around the rounded edges (30B) and/or end (30A) surfaces and onto the inward facing surface 30D, so that the electrodes have a three dimensional curved configuration. As discussed above, the curvature and location of the electrodes of this embodiment of the device will tend to exert higher pressure against the inner surface of the skin or other overlying tissue than does the adjacent relatively flattened outward facing surface of the device.

Because the electrodes extend circumferentially around the end and/or curved sides of the device, they will similarly provide enhanced outward facing contact pressure if the device is flipped over within the pocket. As discussed above, implant of device with this configuration may preferably occur in a location in which a layer of fatty tissue is available to space the electrodes 32 from underlying muscle tissue. As with the electrodes of the device illustrated in FIG. 1, in some embodiments the portions of the electrodes 32 overlying the generally flat outward facing surface 30C of the device may also be rounded or domed as generally illustrated in FIG. 4.

1. An implantable ECG monitor of the type comprising circuitry for monitoring ECGs within a device housing and electrodes located on the exterior of the housing, wherein:
said device housing comprises an elongated housing having generally flat outward and inward facing surfaces and rounded edge and side surfaces; and wherein
a said electrode is located on the outward facing surface of the housing adjacent a said end surface and projects from the facing surface of the housing.

2. An implantable ECG monitor of the type comprising circuitry for monitoring ECGs within a device housing and electrodes located on the exterior of the housing, wherein:
said device housing comprises an elongated housing having generally flat outward and inward facing surfaces and rounded edge and side surfaces; and wherein
a said electrode is located on the outward facing surface of the housing and projects outward from the facing surface of the housing.

3. An implantable ECG monitor of the type comprising circuitry for monitoring ECGs within a device housing and electrodes located on the exterior of the housing, wherein:
said device housing comprises an elongated housing having generally flat outward and inward facing surfaces and rounded edge and side surfaces; and wherein
a said electrode is located on the upward facing surface of the housing adjacent a said end surface and extends onto outward facing portions of the end and/or side surfaces.

4. An implantable ECG monitor of the type comprising circuitry for monitoring ECGs within a device housing and electrodes located on the exterior of the housing, wherein:
said device housing comprises an elongated housing having generally flat outward and inward facing surfaces and rounded edge and side surfaces; and wherein
a said electrode is located on the outward facing surface of the housing and extends onto facing portions of the end and/or side surfaces.

5. A monitor according to claim 3 or claim 4 wherein a said electrode is located on the outward facing surface of the housing and projects outward from the facing surface of the housing.

6. A monitor according to claim 3 or claim 4 wherein a said electrode extends onto the inward facing surface of the housing.
